(19) United States
(12) Patent Application Publication
(10) Pub. No.: US 2021/0137401 A1
(43) Pub. Date: May 13, 2021

(54) MAINTAINING SURFACE MOISTURE TO AID IN ACQUIRING A CONSISTENT GROUND DURING BIO-CONDUCTANCE TESTING

(71) Applicant: Vine Medical LLC, Saint George, UT (US)

(72) Inventor: Phillip W. Dietz, Saint George, UT (US)

(73) Assignee: Vine Medical LLC, Saint George, UT (US)

(21) Appl. No.: 17/091,785

(22) Filed: Nov. 6, 2020

Related U.S. Application Data
(60) Provisional application No. 62/932,374, filed on Nov. 7, 2019.

(51) Int. Cl. A61B 5/0408

(52) U.S. Cl. CPC A61B 5/04087; A61B 2562/0209

(57) ABSTRACT

The disclosure extends to methods, systems, and devices for determining and maintaining a consistent ground and ground saturation resistance in bioelectrical measurements and bio-conductivity testing. The disclosure also extends to methods, systems, and devices for applying and maintaining moisture to aid in acquiring a consistent ground reference during bio-conductance testing. The disclosure includes a conductive device configured to contact skin of a test subject during bioelectric testing. The conductive device itself includes a first conductive portion that is electrically conductive, one or more apertures formed through the first conductive portion, and moistening media that applies a conductive fluid to the skin of the test subject through the one or more apertures.

### CROSS-REFERENCE TO RELATED APPLICATIONS

This application claims the benefit of U.S. Provisional Application No. 62/932,374, filed Nov. 7, 2019, titled, “METHODS, SYSTEMS. AND DEVICES FOR MAINTAINING MOISTURE TO AID IN ACQUIRING A CONSISTENT GROUND DURING BIO-CONDUCTANCE TESTING,” which is incorporated herein by reference in its entirety, including but not limited to those portions that specifically appear hereinafter, the incorporation by reference being made with the following exception: In the event that any portion of the above-referenced provisional application is inconsistent with this application, this application supersedes the above-referenced provisional application.

### TECHNICAL FIELD

The disclosure is directed to obtaining bioelectric measurements using an electrodermal probe in contact with body tissue of a test subject and is particularly directed to methods, systems, and devices for applying and maintaining surface moisture on a test subject to aid in acquiring a consistent ground reference during bio-conductance testing and/or obtaining bioelectric measurements.

### BACKGROUND

The electrical conductance of body tissue can be measured and analyzed to gather information about a body's condition and to aid in assessing certain conditions. One form of measuring electrical conductance of body tissue is Electroacupuncture According to Voll (EAV). EAV and other electrical conductance assessment systems measure conductance levels at meridian points of the body. These electrical conductance assessment systems are used by some health practitioners to gain additional insight into a test subject's body make up and condition.

EAV and other electrical conductance assessment systems often utilize an electrodermal probe in contact with the body tissue of a test subject in order to measure and/or test electrical conductance of the test subject's body tissue at meridian points of the body. During measuring or testing of electrical conductance of the test subject's body tissue, EAV and other electrical conductance assessment systems often utilize a grounding device that contacts the test subject to serve as the ground reference in a test circuit formed using the test subject, and the electrodermal probe and grounding device of the electrical conductance assessment system (e.g., EAV).

Meridian points are located under the skin of a test subject which adds to the difficulty in measuring resistance because skin can become dry and act as an insulator instead of a conductor, thereby impeding electrical conductance and compromising accuracy of readings taken of the meridian points of the test subject. To minimize this insulator effect, several methods may be utilized, either alone or in combination including spraying or applying water or other conductive fluid on the portion of the test subjects skin that contacts the grounding device to help increase the conductivity of the test subjects body tissue. Grounding device is the ground or reference in the test circuit.

In light of the foregoing, disclosed herein are systems, methods, and devices for achieving and maintaining surface moisture on the skin of a test subject in order to improve grounding of the test subject's body tissue during electrical conductance assessment testing.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1 illustrates an example system for obtaining bioelectrical measurements with a digital signal, including an electrodermal probe, a hand mass, and a bioelectrical measurement system and device.

FIGS. 1A-1F illustrate a top, front, side, bottom, top perspective, and rear views of a closed hinged grounding device, respectively.

FIGS. 2A-2D illustrate a top, side, top perspective, and front views of an open hinged grounding device, respectively.

FIGS. 3A-3C illustrate side views of a hinged grounding device with a cover in a closed position, an open position, and with the cover removed from the grounding device, respectively.

FIGS. 4A-4B illustrate an exploded perspective bottom view and a perspective bottom view of a hinged grounding device, respectively.

FIGS. 5A-5B illustrate a perspective view of an open and closed top swinging compartment door, respectively.

FIGS. 6A-6B illustrate two different perspective views of a bottom swinging compartment door in an open position and a bottom swinging compartment door in a closed position, respectively.

FIG. 7 illustrates a top view of a rounded bioelectrical ground plate.

FIG. 8 illustrates a side view of a cylindrical bioelectrical ground plate.

FIG. 9 illustrates a perspective view of a hinged grounding device.

FIG. 10 illustrates an example system for obtaining bioelectrical measurements with a digital signal, including an electrodermal probe, a bioelectrical measurement system and device, and a grounding device.

FIG. 11 illustrates a method of determining a conductance level between a test subject whose skin is in contact with grounding device including grounding plate and grounding segments.

FIG. 12 illustrates a method of performing bioelectric conductance testing using a system including grounding device.

### DETAILED DESCRIPTION

Disclosed herein are systems, methods, and devices, for achieving and maintaining surface moisture on the skin of a test subject in order to improve grounding of the test subject's body tissue during electrical conductance assessment testing. The disclosure extends to systems, methods, and devices, that could be utilized to apply and maintain moisture on a surface of a test subject's body tissue, such as skin, that is in contact with a portion of an electro-conductance testing device such as a test probe tip, hand mass, or grounding device used to ground the test subject. The systems, methods, and devices may be used in conjunction with an electrical conductance assessment system such as an Electroacupuncture According to Voll (EAV) or other electrodermal sensor system.

In the following description, for purposes of explanation and not limitation, specific techniques and embodiments are set forth, such as particular techniques and configurations, in order to provide a thorough understanding of the system and device disclosed herein. While the techniques and embodiments will primarily be described in context with the accompanying drawings, those skilled in the art will further appreciate that the techniques and embodiments may also be practiced in other similar devices.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the systems, methods, and devices, for achieving and maintaining surface moisture on the skin of a test subject in order to improve grounding of the test subject's body tissue during electrical conductance assessment testing are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Electroacupuncture According to Voll (EAV) devices may be deployed to measure conductance levels at meridian points in a body. An EAV device is a sensitive ohm meter for measuring resistance in the body. The resistance of a material, tissue, meridian pathway, and so forth can be assessed to calculate the conductivity of the material, tissue, and/or meridian pathway. A material with a lower resistance measurement will have a higher conductivity.

To detect resistance, an ohm meter (such as an EAV device) applies a small direct current flow through a material. Resistance measures the relative difficulty for current to flow through the material. Electrical conductors allow current to flow easily and have a correspondingly low resistance. Electrical insulators restrict current flow through and have a correspondingly high resistance. Ohm's Law applies to materials with a proportional relationship between voltage, current, and resistance according to:

V=IR

where V is voltage (measured in volts), I is current (measured in amps) and R is resistance (measured in ohms). Conductivity is the reciprocal of resistivity, expressed mathematically as 1/R and indicates a degree to which a specified material conducts electricity.

Human tissue generally has a resistance of about 98,000 Ohms between the tissue and ground. Meridian points have a general resistance of about 5,000 Ohms between the meridian point and ground. This means that meridian points throughout the human body are about twenty times more conductive than the tissue surrounding the points. This large differential in conductivity makes it possible to locate meridian points and to be very consistent in verifying the meridian points with an EAV device.

Traditional EAV testing or resistance measuring of meridian points may be done by having a subject grip a metal rod (hand mass or grounding device) in one hand while point measurements are taken on the other hand and the other side of the body with a probe of the EAV device. The metal rod may be placed in the other hand while the points on the other side of the body are read with the EAV device. The point measurements may use an electrically conductive tip to contact the meridian point and the measurements are taken and recorded while pressure is applied to the metal tip against the tissue at that point.

Meridian points are located under the skin which adds to the difficulty of measuring resistance associated with the meridian point because skin can become dry and act as an insulator. To alleviate this insulating effect, spraying a mist of water or other conducting fluid on the hand that grips the hand mass may be done to help increase the conductivity of the tissue that is gripping the hand mass or metal rod. The hand mass acts as a ground or a ground reference in the test circuit of the conductance testing device. Other methods to decrease this insulating effect is to increase the pressure of the tip against the tissue over the Meridian point, to add moisture or water to the tissue where the measurement is taken, and to apply texture to the tip surface that helps penetrate the insulation layer or a cornified layer of the tissue without puncturing.

In some cases, small electrodes similar to those used with EKG machines in the place of the brass rod or hand mass. Additionally, different sized hand masses may be made to fit different sized hands whether they be a small children or large adults. In testing, it was determined that that the grounding or reference surfaces having larger sizes improved the grounding of the test subject and led to more consistent and more accurate measurements.

Metal rods, handles, hand masses, and/or grounding devices are often implemented using brass due to its relatively high conductivity. However, brass may tarnish and become soiled with use by repeated gripping. The tarnishing may be aggravated by continuing to use the same grounding surfaces, hand mass, and testing tips for multiple patients. Continued and repeated use of these metal rods, handles, hand masses, grounding surfaces, grounding devices, etc. may create oxidation and tarnishing and may degrade the conductivity of the grounding surfaces, hand masses, grounding devices, and test tips associated with the system and device. Therefore, the grounding surfaces, hand masses, grounding devices, and test tips may require frequent cleaning to maintain desired conductivity. However, using a non-oxidizing material or a new tip or grounding device for each new patient may help increase patient to patient measurement accuracy and device cleanliness.

Even with clean surfaces or new conductive material, the moistening of the grounding area of the hand or skin portion being grounded may aid in achieving more accurate and more consistent measurements. Moistening of the surface/skin being grounded may be achieved by frequently squirting a mist of water or other conducting fluid on the surface/skin to keep the skin moist. Skilled technicians may identify when measurements from the conductance measuring device seem to be losing accuracy and may then take action remoisten the subject's skin to achieve improved grounding and more consistent measurements during electro-conductance testing of a test subject.

In order to avoid pausing testing to rewet a test subject's skin with a mist or spray, the grounding device described herein may include a moistening media and apertures in a grounding plate that continually moistens the test subjects skin while in contact with the grounding plate. This configuration has an advantage of maintaining moisture on a test subject during electro conductance testing in order to obtain more consistent and reliable conductance measurements without the need to pause a current test.

The following disclosure is a conductive device configured to contact skin of a test subject during bio-conductance testing of the test subject. The conductive device may comprise a first conductive portion that is electrically conductive, one or more apertures formed through the first conductive portion, and moistening media that applies a conductive fluid to the skin of the test subject through the one or more apertures. The conductive device may be a grounding device for grounding the test subject.

As disclosed herein, the conductive device further includes a cover and a base. The cover is configured to alternate between an open position and a closed position relative to the base. The first conductive portion is disposed on the cover. The moistening media is disposed adjacent to the apertures in the first conductive portion of the cover and between the cover and the base. Furthermore, the cover may comprise an openable and closeable compartment for holding the moistening media adjacent to the apertures of the first conductive portion.

The base may comprise one or more second conductive portions. Furthermore, the cover is in electrical communication with the base when the cover is in the closed position and the cover is electrically separated from the base when the cover is in the open position. One or more second conductive portions are disposed on the base such that the one or more second conductive portions contact a palm of a hand of the test subject during the bioelectric testing, and the first conductive portion is disposed on the cover such that the first conductive portion contacts fingers and/or palm of the hand of the test subject during the bioelectric testing. The cover is in electrical communication with the base through a conductive contact on the base that contacts the first conductive portion of the cover. The conductive contact is spring-biased to press against the first conductive portion when the cover is in the closed position.

The base of the conducting device may comprise one or more of a battery to power the conductive device and an electrical connection to receive electricity from an outside power source to power the conductive device.

The apertures of the conductive device may be circular, or the apertures of the conductive device may be slits.

The base further includes a palm locating rib indicating where a test subject's palm may be placed on the conductive device. The conductive device described herein the first conductive portion is substantially plate-shaped. The conductive device described herein the first conductive portion is substantially bar-shaped.

The base includes a pin, the cover includes a knuckle that engages with the pin to form a hinge, and the cover rotates about the hinge to alternate between the open position and the closed position.

Part of the disclosure includes a bioelectric testing system for taking bioelectric measurements of a test subject. The bioelectric testing system includes a grounding device for contacting a first portion of a test subject. The grounding device includes a first conductive portion that is electrically conductive and contacts skin of the test subject, one or more apertures formed through the first conductive portion, and moistening media that applies a conductive fluid to the skin of the test subject through the one or more apertures. The system may further include an electrodermal probe for contacting a second portion of the test subject including a probe tip that contacts the second portion of the test subject.

Now referring to the figures, FIG. 1 illustrates a perspective view of a bioelectric measurement system 10, which is an example of an EAV or electrical conductance assessment system. As shown, bioelectric measurement system includes an electrodermal probe 22, grounding device 30 (e.g., a hand mass, or grounding surface), and a bioelectric measurement analyzing device 18 in electrical communication with electrodermal probe 22 and grounding device 30.

Grounding device 30 may include a handheld mass to be held by a test subject undergoing measurements by bioelectric measurement system 10. Grounding device 30 may include a rod made of brass, or any other suitable material for grounding the test subject. Grounding device 30 may include a grounding surface disposed around an exterior of grounding device 30.

Grounding device 30 may be a small electrode similar to those used in conjunction with an electrocardiogram (EKG). Grounding device 30 may be any suitable size or shape and may be formed in an ergonomic size and shape that is easy for a test subject to hold in a palm of the test subject's hand. Grounding surface of grounding device 30 may be of a sufficiently large size to provide ample grounding to take sufficiently consistent and sufficiently accurate measurements from the test subject.

Electrodermal probe 22 is configured to measure the resistance of skin, a meridian pathway in a body, or other materials or tissues. The readings taken by electrodermal probe 22 can be assessed to calculate the conductivity of the skin, the meridian pathway in the body, or other materials or tissues in the body. Electrodermal probe 22 may include a testing end, which may include a probe hood 24 and a probe tip 26 disposed at a distal end, or in other words testing end, of electrodermal probe 22 with respect to the electrical connection with bioelectric measurement analyzing device 18. Probe tip 26 may be placed against the skin of a test subject to enable electrodermal probe 22 to measure the resistance of the skin or meridian pathway in the test subject. The electrodermal probe 22 may take a measurement when probe tip 26 is pressed against tissue. Probe tip 26 may be constructed of any suitably electrically conductive material such as copper, silver, gold, aluminum, zinc, nickel, brass, iron, steel, or other material known to those skilled in the art.

The probe tip 26 may be a single probe tip or the probe tip 26 may include a plurality of individual probe tips. Probe tip 26 may be textured to help penetrate and help electricity flow through the insulation layer or cornified layer of the epithelial tissue without puncturing it. The grounding pads and/or contacts may be integrated with probe hood 24. Electrodermal probe 22 may further include a contact sensor 28 disposed on probe hood 24 to detect proper contact between probe tip 26 and the test subject.

The bioelectric measurement analyzing device 18 may include one or more processors configurable to execute instructions stored in non-transitory computer readable storage media. Bioelectric measurement system 10 may include memory stored locally therein and accessible by bioelectric measurement analyzing device 18. Bioelectric measurement analyzing device 18 is in electrical communication with electrodermal probe 22, grounding device 30, and a display 16. In the illustration shown in FIG. 1, bioelectric measurement analyzing device 18 is in electrical communication with electrodermal probe 22 by way of a sensor connection point 14 and is in electrical communication with grounding device 30 by way of grounding connection point 12. The electrical communication between bioelectric measurement analyzing device 18 and electrodermal probe 22 and/or grounding device 30 can be facilitated by electrically conductive cables 20. Alternatively, the electrical communication may be made wirelessly through a wireless network such as a wireless personal area network (WPAN), a wireless local area network (WLAN), and so forth. Electrically conductive cable 20 may further be connected to a power source such that electrodermal probe 22 and/or grounding device 30 are powered by way of an external power source.

Conductance testing or measuring of meridian points may be done by having a test subject grip a conductive rod or hand mass (grounding device 30) in one hand while point readings are taken on the other hand and or the other side of the body using electrodermal probe 22. Then the conductive rod (grounding device 30) may be placed in the other hand while the point readings are taken on the other side of the test subject's body with electrodermal probe 22. Electrodermal probe 22 may utilize probe tip 26 to contact the meridian points, and the readings may be taken and recorded while pressure is applied against the tissue at that point.

The grounding device 30 shown in FIG. 1 is an example of a grounding device that may be used in conjunction with bioelectric measurement system 10. Several other configurations of grounding devices may be used in conjunction with bioelectric measurement system 10. Multiple embodiments of grounding devices that may be used in place of grounding device 30.

A grounding device 100 is shown and described in FIGS. 1A-1F. Illustrated in FIGS. 1A-1F are top (FIG. 1A), front (FIG. 1B), side (FIG. 1C), bottom (FIG. 1D), perspective (FIG. 1E), and rear/back (FIG. 1F) views of a hinged grounding device 100 in a closed position. As illustrated in FIGS. 1C and 1E, grounding device 100 may include a cover 102 and a base 114. Cover 102 may include a knuckle 104 that engages with a pin 116 on base 114 to form hinge H. With knuckle 104 engaged with pin 116, cover 102 may rotate about pin 116 with knuckle 104 to rotate between an opened and a closed position, which will be described in more detail in the discussion of FIGS. 3A-3C in this disclosure. FIGS. 1A-1F show a configuration of grounding device 100 where cover 102 is in the closed position.

The grounding device 100 may incorporate one or more conductive portions. For example, cover 102 of grounding device 100 may include a grounding plate 108. Grounding plate 108 may have one or more apertures 110 formed therein to allow materials and fluids to permeate through grounding plate 108 of cover 102 to moisten the skin of a human test subject in contact with grounding plate 108. The size and shape of grounding plate 108 is not particularly limited as long as grounding plate 108 is of sufficient size to provide proper grounding for the test subject. For example, grounding plate 108 may be around eight square inches, but may be larger or smaller depending on characteristics of the test subject and/or other circumstances such as, for example, environmental conditions and/or the level of moisture applied to skin of the test subject. Grounding plate 108 may be composed of brass or stainless steel, but it will be appreciated that grounding plate 108 may be composed of any electrically conductive material that allows electricity to flow in one or more direction.

Additionally, grounding plate may further be any mesh screen, perforated metal, porous materials, or any conductive material that allows moisture of a conducting fluid to pass through to a skin surface of a test subject while electrically grounding the test subject.

As shown in various figures of FIGS. 1A-1F, grounding device 100 may include a plurality of conductive portions. One conductive portion may be grounding plate 108 disposed on cover 102. Additionally, one or more additional conductive portions (grounding segments 122A and 122B, which may be collectively referred to as grounding segments 122) may be disposed on base 114. Grounding segments 122 may contact a portion of the palm of the hand such as the heel of the palm. The rest of the hand (e.g., other portions of the palm, and/or fingers) may contact grounding plate 108. The bioelectrical grounding device 100 may include a palm locating rib 120 on the proximal end of a base 114. One or more grounding segments 122 may be disposed distally with respect to a palm locating rib 120. As shown in at least FIGS. 1C, 1E, 3A, and 9, grounding plate 108 and base 114 may be curved to fit a curve of a test subject's hand including the palm and fingers for ergonomic comfort and maintenance of reliable contact between the test subject and the grounding device when testing is carried out.

As shown in at least FIGS. 1A-1F, grounding device 100 may include two conductive portions in addition to grounding plate 108. A conductivity test, which may be separate from the conductance testing done on meridian points, may be performed on the test subject to check conductance levels and/or ground saturation before performing conductance testing on meridian points and/or meridian pathways. The conductivity test may be performed by taking a first measurement between one of the two conductive portions (e.g., grounding segments 122A and 122B) and grounding plate 108. After the first measurement, the other grounding segment 122A or 122B (e.g., the grounding segment not used in the first measurement) of grounding segments 122 may be added in series with the grounding plate 108 to facilitate performing a second measurement. This second measurement may be compared with first measurement to determine a level of ground saturation. If both the first and second measurements are the same, then it may be determined that an adequate ground saturation condition is achieved. If the one of the measurements has a greater conductivity measurement when compared with the other measurement, then adequate ground saturation is not achieved, which could result in inaccurate and inconsistent measurements. This situation may usually be remedied by moistening the grounding hand with more conducting fluid or a mist of conducting fluid sprayed on the grounding hand to help reestablish a ground saturation condition.

### Ground Saturation Testing

To better understand and illustrate ground saturation the following test was performed. A large hand mass, which was a brass rod (1 inch diameter×3.4 inches long), was covered with strips of nonconductive tape that wrapped around a diameter or perimeter of the brass rod such that each strip of tape covered five percent (5%) of the brass rod or hand mass. A conductance test was carried out using a test subject gripping the hand mass with a dry hand. With 20 strips of tape on the rod the hand mass was completely covered rendering the entire rod nonconductive. Under these conditions, the system was unable to take a measurement.

Next, a first strip of tape was removed exposing five percent (5%) of the conductive brass rod, which resulted in a successful conductance measurement being taken. The surface area of the brass rod is about ten square inches and each strip of tape covered about 0.5 square inches of surface area on the brass rod. Each strip of tape covering the rod were removed one at a time, thus exposing an additional 0.5 square inches of the brass rod, with a conductance measurement being taken each time a strip of tape was removed. As each strip of tape was removed, the conductivity measurement increased because the ground area increased. At the same time as each successive piece of tape was removed, the successive incremental increase in conductance decreased until the eighth strip of tape was removed and about forty percent (40%) of the conductive area or about four square inches of the brass rod was exposed. With about forty percent (40%) of the conductive area exposed, the conductivity was stable and no longer changing upon testing at the same point. With the conductivity stable, the measurement accuracy then remained the same after this point even as more tape was removed and even until the entire brass was exposed. Accordingly, about forty percent (40%) or about four square inches of the surface area of the brass rod exposed was determined to provide sufficient ground saturation.

### Effect of Moisture on Ground Saturation

Additional testing was carried out using the same tape removal test described above. In the additional testing, a test subject gripped the hand mass with a wet hand. The additional testing shows that moisture applied to the skin during conductance testing affected the amount of surface area on the brass rod required to establish sufficient ground saturation. The testing revealed that only about 2.8 square inches of ground area was required to establish ground saturation with a wet hand instead of the about four square inches required for achieving sufficient ground saturation with a dry hand. Accordingly, a lower area was need for ground saturation to be achieved with a wet hand than with a dry hand. A required area for ground saturation may vary from person to person based upon the person's skin conductivity and the other factors, such as age, gender, ethnicity, geography, atmospheric, environmental conditions, and the like.

Accordingly, because adding moisture or a fluid between a hand and a ground provided increased accuracy and ground saturation with a smaller surface contact area, bioelectrical grounding device 100 allows moisture to continually permeate through grounding plate 108 using apertures 110 to moisten the hand throughout the duration of a bioelectric measurement test and conductance testing. The bioelectrical grounding device 100, device 100 may include a brass mesh material as at least part of grounding plate 108 with a moistening media, which may be, for example, a wet sponge applied to grounding plate 108 to supply continual moisture to skin of a test subject through apertures 110 in grounding plate 108 for increasing conductivity and maintaining ground saturation for a duration longer than a typical test period.

Grounding device 100 and or grounding plate 108 may be implemented as a perforated stainless sheet to address bending, forming and cleanliness issues.

Moistening media (shown later) functions to hold water or other conducting fluids to apply continuous moisture to grounded skin of a test subject. The moistening media may be a pad, a sponge, a cloth, towel, rag, or any material suitable for releasing moisture or fluid onto a test subject's skin. Other moistening media may be implemented such as a vaporizer, mister, atomizer, sprayer, dropper, pumps, or other device for dispensing moisture/fluid on to a test subject's skin through grounding plate 108. In testing, the moistening media may be implemented as a compressed type of sponge that is initially 1/16 inch thick. When moistened, the 1/16 inch moistening media expands to approximately ⅜ inch thick, which filled its moistening potential and expanded against apertures 110 in grounding plate 108. Thus, the moistening media expanded through apertures 110 and applied ample moisture to the contacting surface of the hand of the test subject. Many different sizes and types of material may be used to maintain continuous moisture to the skin through apertures 110 and may act as the moistening media. The metal used in testing of grounding plate 108 has a thickness of about 0.024 inch with about 0.08 inch diameter for apertures 110. The open perforation density was about thirty-five percent (35%) leaving about sixty-five (65%) of conductive metal contacting the skin for grounding. It will be appreciated that grounding plate 108 may be made from many electrically conductive materials, having a number of different thicknesses, sizes, and geometric shapes without departing from the scope of the disclosure. Furthermore, apertures 110 may be various sizes, shapes, thicknesses, and perforation density, such that apertures 110 allow sufficient moistening fluid to penetrate grounding plate 108 to moisten the skin of the test subject.

To aid in consistency and cleanliness, moistening media may be a disposable item (e.g., easily removed and replaced by a new moistening media). It is further conceivable that that grounding plate 108 and the moistening media may both be disposable (e.g., one time use items) that are pre-moistened with water or another conducting fluid. The conducting fluid may provide more accurate test results if it is formulated with a suitable PH balance and with a controlled conductivity as to be compatible with human tissue of all types. The conducting fluid may aid in normalizing the tissue conductivity from patient to patient.

The grounding plate 108 may be formed to fit a wide variety of hands comfortably. Grounding plate 108 may also be attached to a cover 102 that may further be attached and detached from the base 114 of bioelectrical grounding device 100. Cover 102 may be made of a rigid structure and may include grounding plate 108 and apertures 110 which allow moisture to be dispersed by the moistening media to the test subject. Cover 102 may be hinged, and attached, to base 114 by at least one knuckle 104, slot 106, and pin 116 (e.g., hinge H). Grounding plate 108 may be implemented with apertures 110 and hinge H on cover 102 such that grounding plate 108 opens in an upward direction to expose moisture compartment (shown in at least FIG. 5A) for easy removal and replacement of the moistening media. In this manner a technician may easily change the moistening media with a new moistening media for each new patient.

The cover 102 may comprise one or more knuckles 104 including a slot 106. Knuckle 104 disposed on cover 102 and slot 106 facilitates removal of cover 102 from base 114. The corresponding base 114 may include a pin 116 that may be secured in position by one or more removal guides 118. As knuckle 104 pivots around pin 116, slot 106 aligns with removal guides 118 and knuckle 104 may slide off pin 116 allowing cover 102 to be separated from base 114 (shown in further detail with respect to FIGS. 3A-3C. Because cover 102 may be removed from bioelectrical grounding device 100, bioelectrical grounding device 100 may still allow moisture to be applied to the moistening media while cover 102 is removed from base 114. Cover 102 may then be placed back onto base 114 in a moistened state to help keep extra unwanted water/conducting fluid from compromising electronics and other integral components.

Bioelectrical grounding device 100 may include a spring-loaded contact that engages with a cover latch 128 on base 114 when grounding plate 108 is properly assembled and secured to bioelectrical grounding device 100. Bioelectrical grounding device 100 may include a cord or a battery to power electrical power to bioelectrical grounding device 100. Switch 130 may have various functions that include being an on/off switch or it may activate a ground saturation test etc. Switch 130 may be located at the side of base 114 in the middle of pin 116 as shown and the battery may be disposed in the bottom of the base 114. The position of switch 130 is not particularly limited and maybe placed anywhere on base 114 or cover 102 of grounding device 100 including between base 114 and cover 102. Access to the battery may be from the bottom of base 114 through a battery plate 124 that includes a battery plate latch 126.

Illustrated in FIGS. 2A-2D are top (FIG. 2A), side (FIG. 2B), perspective (FIG. 2C), and rear/back (FIG. 2D) views of grounding device 100 where cover 102 is in the open position. The bioelectrical grounding device 100 shown in FIGS. 2A-2D may include a cover latch 128 on an end of base 114. Cover latch 128 may lock cover 102 in the closed position to base 114 as shown in FIGS. 1A-1F. As shown in FIGS. 2A-2D cover latch 128 may be actuated to release cover 102 and to allow cover 102 to pivot to the open position.

The grounding plate 108 may be composed of stainless steel, but it will be appreciated that grounding plate 108 may be composed of any electrically conductive material that allows electricity to flow in one or more direction.

As illustrated in FIGS. 2A-2D, grounding plate 108 may include a cover grounding contact 134 that may be located on cover 102. As shown in FIGS. 2A-2D, grounding device 100 may further include a spring-loaded grounding contact 133 on base 114. Grounding contact 133 corresponds with cover grounding contact 134 on grounding plate 108 of cover 102. Grounding contact 133 located on base 114 may be spring-loaded to be biased to press against and contact cover grounding contact 134 on cover 102 for electrically connecting cover 102 to the test circuit. Grounding contact 133 engages with a cover grounding contact 134 on cover 102 when cover 102 is in the closed position and secured to base 114 with cover latch 128 of bioelectrical grounding device 100.

FIG. 2B illustrates a side view of bioelectrical grounding device 100 and shows a side view of both base 114 and cover 102. Base 114 includes the one or more grounding segments 122 and cover latch 128 on the distal end of base 114 that secures cover 102 in a closed position. Further on the side of the proximal end of base 114 is pin 116. Pin 116 includes one or more removal guides 118 described in more detail with respect to FIGS. 3A-3C. Slot 106 constitutes a portion of knuckle 104 that is open to allow cover 102 to be removed from removal guides 118 to allow cover 102 to be detached from base 114. Hinge H that includes knuckle 104, slot 106, pin 116 and removal guides 118 may be located on both sides of bioelectrical grounding device 100.

FIG. 2C illustrates palm locating rib 120 on the proximal end of base 114. Grounding segments 122A and 122B may be disposed distally to palm locating rib 120 as illustrated. On the distal end of base 114 is a cover latch 128. Cover latch 128 may lock cover 102 to base 114. As shown in FIG. 2C, an underside of cover 102 shows an underside of a compartment that holds moistening media 116 and opens to allow installation of a moistening media 116 therein. The compartment further includes one or more latches 112 that operate to hold the compartment shut and may be released to allow compartment door 132 (shown and described with respect to FIGS. 5A and 5B) to open when latches 112 are unlatched. The perspective view of FIG. 2C further illustrates hinge H including pin 116 and knuckle 104, slot 106, about which cover 102 rotates between the open and closed position.

FIG. 2D illustrates a rear view of a hinged bioelectrical grounding device 100 in an open position. FIG. 2D illustrates palm locating rib 120 on the proximal end of base 114. Grounding segments 122A and 122B are shown disposed distally with respect to palm locating rib 120. Grounding plate 108 is disposed on the cover 102 and includes a plurality of apertures 110. The rear view of FIG. 2D also illustrates a battery plate latch 126 that aids to secure battery plate, which is described and shown in further detail with respect to FIGS. 4A and 4B.

FIGS. 3A-3C illustrate side views of a cover 102 in a closed position (FIG. 3A), an open position (FIG. 3B), and being removed (FIG. 3C) from a base 114. FIG. 3A illustrates cover 102 of bioelectrical grounding device 100 in a closed position. Grounding device 100 includes base 114 that is composed of a rigid material. Cover 102 is connected to base 114 at hinge H. While in a closed position, cover 102 may be secured to base 114 by cover latch 128. Further illustrated in FIG. 3A is a side view of a palm locating rib 120 located on a proximal end of base 114 and one or more grounding segments 122 disposed distally with respect to palm locating rib 120.

Also shown in FIG. 3A, on the side of the proximal end of base 114, is pin 116 including removal guides 118. Removal guides 118 may include two flat surfaces on the top and bottom of pin 116. Slot 106 may be an opening in knuckle 104 of cover 102. Hinge H may include knuckle 104, slot 106, pin 116 and removal guides 118. Hinge H may be located on each side of bioelectrical grounding device 100. When cover latch 128 is released, cover 102 may rotate about pin 116 from the closed position to the open position.

FIG. 3B illustrates bioelectrical grounding device 100 where cover 102 of bioelectrical grounding device 100 is in the open position. As shown in FIG. 3B, cover 102, including grounding plate 108, is separated from base 14 and cover grounding contact 134 of grounding plate 108 is no longer in contact with grounding contact 133 of base 114. Accordingly, in the open position, cover 102 may be electrically separated from base 114.

Furthermore, when cover 102 is in the open position, knuckle 104 and slot 106 are in a position with respect to removal guides 118 and pin 116 to be removed from pin 116 and base 114. As illustrated in FIG. 3B in conjunction with FIG. 3C, pin 116 includes removal guides 118, which may be two flat surfaces on the top and bottom of pin 116. Left and right sides of pin 116 have rounded surfaces 140 having a radius substantially matching an inner radius of knuckle 104 on cover 102. A distance between the flat surfaces of removal guides 118 substantially matches a distance of slot 106. Accordingly, cover 102 may be engaged with pin 116 of base 114 by sliding knuckle 104 over pin 116 in the open position of cover 102. A distance between rounded surfaces 140 is larger than a distance between removal guides 18 such that, as cover 102 is rotated from the open position (FIG. 3B) down to the closed position (FIG. 3A), slot 106 is no longer aligned with removal guides and is instead firmly held to be engaged with pin by rounded surfaces 140 engaged with knuckle 104. In other words, slot 106 constitutes a portion of knuckle 104 that when rotated with respect to pin 116 and removal guides 118 allow cover 102 to detach from base 114.

FIG. 3C illustrates cover 102 of bioelectrical grounding device 100 removed from base 114. As shown in FIG. 3C, bioelectrical grounding device 100 includes palm locating rib 120 disposed on the proximal end of base 114.

FIGS. 4A-4B illustrate an exploded bottom perspective view (FIG. 4A) and a bottom perspective view (FIG. 4B), respectively, of hinged grounding device 100. FIG. 4A illustrates a battery plate 124 that may be removably attached to base 114. Battery plate latch 126 may be attached to battery plate 124 and may be operated to remove battery plate 124 from base 114. A battery 136 may be placed in battery compartment or recess 138 that may be formed within base 114. Base 114 may be composed of a rigid material and is connected to cover 102.

FIG. 4B illustrates battery plate 124 attached to base 114 in a closed position. Battery latch 126 that is attached to battery plate 124 may be used for securing battery plate 124 to base 114. The battery (not shown) may reside beneath battery plate 124 and may be placed in battery compartment 138 disposed within base 114.

FIG. 5A illustrates a perspective view of a cover 102 with a grounding plate 108 in an open position. As illustrated, grounding plate 108 may also operate as a compartment door for a compartment that holds moistening media 115 in place within cover 102. Grounding plate 108 may be secured to cover 102 in such a manner so as to open from the top of cover 102 to allow access to a moisture compartment 117. Grounding plate 108 may be secured in a closed position over moisture compartment 117 using latches 112 and may be released from the closed position by operating and releasing latches 112. The number of latches is not particularly limited and may include one or more latches to secure grounding plate 108. Moisture compartment 117 may be used to house a moistening media 115. For example, before testing, water or conducting fluids may be introduced to moistening media 115. Then grounding plate 108 may be moved to a closed position.

The grounding plate 108 may include a plurality of apertures 110 to allow moisture from moistening media 115 to permeate through apertures 110 to the surface of cover 102 and onto skin of a test subject in contact with grounding plate 108. Furthermore, moistening media 115 may be placed in moisture compartment 117, which may be formed as a recess within cover 102. Grounding plate 108 may be closed to secure moistening media 115 inside moisture compartment 117.

Moistening media 115 may be of a size and shape to fit in moisture compartment 117. Additionally, moistening media 115 may be a size that is larger than moisture compartment 117 but may be made of a material that may be compressed to fit into moisture compartment 117, such as a sponge, cloth, towel, rag, or any material suitable for releasing moisture or fluid onto a test subject's skin, etc. In such a configuration, the expanded sponge may expand against apertures 110 and be exposed through apertures 110 in order to better contact skin of a test subject.

Also illustrated in FIGS. 5A and 5B, are knuckles 104A and 104B with corresponding slots 106A and 106B. Knuckles 104A and 104B may be sized and shaped to correspond with a pin 116 attached to a base 114. Likewise, slot 106A and slot 106B correlate with removal guides 118 of pin 116 or pins 116A and 116B, which are attached on opposite sides of base 114.

FIG. 5B illustrates grounding plate 108 in a closed position thereby securing moistening media 115 within moisture compartment 117 (not visible in FIG. 5B). The grounding plate 108 may act as a compartment door and is illustrated in a closed position. Grounding plate 108 may be latched or otherwise secured to cover 102 with a latch 112 to maintain grounding plate 108 in a closed position.

FIGS. 5A and 5B illustrate a configuration of cover 102 where moisture compartment 117 is opened from a top of cover 102 by opening and rotating grounding plate 108 to expose moisture compartment 117. The moisture compartment 117 may be opened from the bottom of cover 102. Additional details will be described with respect to FIGS. 6A and 6B.

FIGS. 6A-6B illustrate two different perspective views of a cover 602 where moisture compartment 117 is in an open (FIG. 6A) and closed (FIG. 6B) position, respectively. FIG. 6A illustrates cover 602 with moisture compartment 617 in an open position. As illustrated, cover 602 may comprise a grounding plate 608 and a compartment door 618 that allows access to an area that houses a moistening media 616. As illustrated, compartment door 618 opens from a bottom position of cover 602. Compartment door 618 may be hinged and may move with respect to cover 602, such that moistening media 616 is accessible from the bottom of cover 602. Moistening media 615 may be located within moisture compartment 617 of cover 602 and compartment door 618 in the closed position may hold moistening media 615 within a moisture compartment 617. When moistening media 615 is located within moisture compartment 617, it is positioned adjacent to grounding plate 608. As noted previously, grounding plate 608 comprises a plurality of apertures 610 to allow moisture to seep through apertures 610 from moistening media 615. Moistening media 615 may be placed in moisture compartment 617. Grounding plate 608 may be closed securing moistening media 615 in moisture compartment 617 and latched with latches 612. Also illustrated, are knuckles 604A and 604B with corresponding slots 606A and 606B. Knuckles 604A and 604B have a size and shape that correlates and corresponds with a pin 116 attached to a base 114 (not shown). Likewise, slots 606A and 606B correlate and correspond with a removal guides 118 of a pin 116 attached to a base 114 (not shown).

FIG. 6B illustrates a perspective view of a cover 602 with a hinged or swinging compartment door 618 in a closed position. Moistening media 615 may be placed in a moisture compartment 617. Compartment door 618 may be closed securing moistening media 615 in moisture compartment 617 and may be latched with latches 612 to maintain moistening media 615 within compartment 617.

FIG. 7 illustrates a top view of a grounding plate 708. Grounding plate 708 may be a rounded shape and may comprise a plurality of apertures 710. Grounding plate 708 may be shaped as a dome and may be attached to a base (not shown). A moistening media (not shown) may be located between the base and grounding plate 708, such that there may be a fluid that flows through apertures 710 to moisten a hand of a user or patient.

FIG. 8 illustrates a side, perspective view of a grounding plate 808 that may be cylindrically shaped. Grounding plate 808 may comprise a plurality of apertures 810. A moistening media (not shown) may be located within the cylindrically shaped grounding plate 808, such that there may be a fluid that flows through apertures 810 to moisten a hand of a user, test subject, or patient holding grounding plate 808.

FIG. 9 illustrates perspective top view of a hinged grounding device 900. Grounding device 900 may comprise cover 902 and a base 914. Cover 902 may be hinged, and attached, to base 914 by way of a hinged mechanism. The hinged mechanism may comprise a at least one knuckle 904 with a corresponding slot 906 and a pin 916 with one or more removal guides 918. It will be appreciated that knuckle 904 and slot have a size and shape that corresponds and correlates with pin 916 and removal guides 918, which may be attached to base 914.

The cover 902 may comprise a grounding plate 908. Grounding plate 908 itself may comprise a plurality of apertures 910. As shown, apertures 910 may have a slit shape instead of a circular shape. The shapes of the apertures are not limited to the configurations shown and described herein. The apertures may be of any shape that allows liquid to pass from the moistening media to the skin of the test subject and may be chosen for utilitarian or decorative purposes, so long as the apertures are of a sufficient size, number, and density to allow conducting fluid to be dispersed therethrough. The plurality of apertures 910 function to allow moisture to pass through grounding plate 908 to the surface, such that there is contact with the skin of the user, test subject, or patient. Grounding plate 908 may be composed of a conductive material, such as stainless steel, but it will be appreciated that grounding plate 908 may be composed of any electrically conductive material that allows electricity to flow in one or more direction.

As illustrated in FIG. 9, a palm locating rib 920 may be disposed on a proximal end of a base 914 as illustrated. One or more grounding segments 922 may be located and disposed distally with respect to palm locating rib 920. A switch 930 may be located distally with respect to the one or more grounding segments 922 or near pin 916. Switch 930 may have various functions that include being an on/off switch or it may activate a ground saturation test etc. The distal end of cover 902 may comprise a cover latch 928. Cover latch 928 may operate to lock cover 902 down to base 914 as similarly described previously.

FIG. 10 illustrates a bioelectric measurement system 10 as shown in FIG. 1. As shown in FIG. 10, grounding device 100 may be used in place of hand mass 30 and may act as the grounding device/hand mass for bioelectric measurement system 10.

FIG. 11 illustrates a method 1100 of determining a conductance level between a test subject whose skin is in contact with grounding device 100 (or any other grounding device embodiment described herein) including grounding plate 108 (with moistening media contained therein) and grounding segments 122A and 122B. As illustrated, in step 1102 of method 1100 a test subject's skin (e.g., hand) is placed in contact with grounding plate 108, grounding segment 122A, and grounding segment 122B. As illustrated, in step 1104 of method 1100 a first conductance reading is taken between a first portion (e.g., a left side of a heel of the palm) of the test subject's skin in contact with grounding segment 122A and a second portion (e.g., fingers/upper palm) of test subject's hand in contact with grounding plate 108. As illustrated, in step 1106 of method 1100 a second conductance reading is taken between the first portion (e.g., a left side of a heel of the palm) of the test subject's skin in contact with grounding segment 122A, a second portion (e.g., fingers/upper palm) of test subject's hand in contact with grounding plate 108, and a third portion (e.g., a right side of a heel of the palm) of the test subject's skin in contact with grounding segment 122B which is added in series with grounding plate 108. As illustrated, in step 1108 of method 1100 the first conductance reading is compared with the second conductance reading to determine if the numbers are sufficiently similar or different from each other. As illustrated, in step 1110 of method 1100, if it is determined that the first measurement and the second measurement are different, then that conductivity/ground saturation is insufficient (e.g., not enough moisture is present over the test subject's hand) and the hand or moistening media is remoistened and the conductance testing is repeated. As illustrated, in step 1112 of method 1100 if it is determined that the first measurement and the second measurement are sufficiently similar then it is determined that conductivity/ground saturation is sufficient (e.g., enough moisture is present over the test subject's hand) to carry out further conductance testing on meridian points of the test subject.

Determining whether the first measurement and the second measurement are sufficiently similar may be done by comparing a difference between the first measurement and the second measurement to a predetermined threshold. If the difference is greater than the predetermined threshold then the measurements are different than each other. If the difference is less than the predetermined threshold then the measurements are substantially similar to each other. Any portion of the method illustrated in FIG. 11 and described above may be carried out by one or more processors configurable to execute instructions stored in non-transitory computer readable storage media.

FIG. 12 illustrates a method 1200 of performing bioelectric conductance testing using a system including grounding device 100, or any other grounding device embodiment described herein. As illustrated, in step 1202 of method 1200 a moistening media is wetted with a conductive fluid and placed adjacent to a grounding plate and apertures of a grounding device. As illustrated, in step 1204 of method 1200 a test subject's skin (e.g., hand) is placed in contact with a grounding plate of grounding device 100 and the moisture on the moistening media. As illustrated, in step 1206, a user takes one or more bioelectric conductance measurements on one or more meridian points of the test subject by contacting the meridian point of the test subject with an electrodermal probe.

### EXAMPLE EMBODIMENT(S)

Example 1 is a grounding device configured to contact skin of a test subject during bioelectric testing of the test subject. The conductive device includes a first conductive portion that is electrically conductive, one or more apertures formed through the first conductive portion, and moistening media that applies a conductive fluid to the skin of the test subject through the one or more apertures.

Example 2 is the grounding device of Example 1, wherein the device is curved to fit the palm of a hand.

Example 3 is the grounding device of Examples 1-2, further comprising a cover, and a base, wherein the cover is configured to alternate between an open position and a closed position relative to the base.

Example 4 is the grounding device of Examples 1-3, wherein the first conductive portion is disposed on the cover.

Example 5 is the grounding device of Examples 1-4, wherein the moistening media is disposed adjacent to the apertures in the first conductive portion of the cover and between the cover and the base.

Example 6 is the grounding device of Examples 1-5, wherein the cover comprises an openable and closeable compartment for holding the moistening media adjacent to the apertures of the first conductive portion.

Example 7 is the grounding device of Examples 1-6, wherein the base comprises one or more second conductive portions.

Example 8 is the grounding device of Examples 1-7, wherein the cover is in electrical communication with the base when the cover is in the closed position and the cover is electrically separated from the base when the cover is in the open position.

Example 9 is the grounding device of Examples 1-8, wherein the one or more second conductive portions are disposed on the base such that the one or more second conductive portions contact a palm of a hand of the test subject during the bioelectric testing, and the first conductive portion is disposed on the cover such that the first conductive portion contacts fingers and/or palm of the hand of the test subject during the bioelectric testing.

Example 10 is the grounding device of Examples 1-9, wherein the cover is in electrical communication with the base through a conductive contact on the base that contacts the first conductive portion of the cover.

Example 11 is the grounding device of Examples 1-10, wherein the conductive contact is spring-biased to press against the first conductive portion when the cover is in the closed position.

Example 12 is the grounding device of Examples 1-11, wherein the base comprises one or more of a battery to power the conductive device and an electrical connection to receive electricity from an outside power source to power the conductive device.

Example 13 is the grounding device of Examples 1-12, wherein the apertures are circular.

Example 14 is the grounding device of Examples 1-13, wherein the apertures are slits.

Example 15 is the grounding device of Examples 1-14, wherein the base further comprises a palm locating rib indicating where a test subject's palm may be placed on the conductive device.

Example 16 is the grounding device of Examples 1-15, wherein the first conductive portion is substantially plate-shaped.

Example 17 is the grounding device of Examples 1-16, wherein the first conductive portion is substantially bar-shaped.

Example 18 is the grounding device of Examples 1-17, wherein the base comprises a pin, the cover comprises a knuckle that engages with the pin to form a hinge, and wherein the cover rotates about the hinge to alternate between the open position and the closed position.

Example 19 is the grounding device of Examples 1-18, further comprising large conductor covers that contacts the hand.

Example 20 is the grounding device of Examples 1-19, further comprising smaller conductors that contact the heel of the hand.

Example 21 is the grounding device of Examples 1-20, wherein the conductive portions maintain a majority of the surface area for grounding.

Example 22 is the grounding device of Examples 1-21, wherein the moistening media includes a sponge, towel, rag, or other material that retains moisture.

Example 23 is the grounding device of Examples 1-22, wherein the moistening media includes a vaporizer, sprayer, droppers, pumps and or any device that can apply moisture to the grounding surface.

Example 24 is the grounding device of Examples 1-23, wherein the cover plate may be removed from the grounding device.

Example 25 is the grounding device of Examples 1-24, wherein the cover plate includes a hinge.

Example 26 is the grounding device of Examples 1-25, further comprising a moistening media compartment.

Example 27 is the grounding device of Examples 1-26, wherein the cover plate is hinged to allow access to a moistening media tray in moistening media compartment.

Example 28 is the grounding device of Examples 1-27, wherein the moistening media tray is hinged to allow access to moistening media.

Example 29 is the grounding device of Examples 1-28, wherein cover plates may be combined with the moisture media to simplify application process and aid in controlling the wetness of the moistening media.

Example 30 is the grounding device of Examples 1-29, wherein the combined cover plates and moistening media are pre-moistened.

Example 31 is the grounding device of Examples 1-30, wherein the combined cover plates and moistening media are disposable.

Example 32 is the grounding device of Examples 1-31, wherein the moistening media is moistened with wetting fluid that is formulated to normalize skin type conditions.

Example 33 is a bioelectric testing system for taking bioelectric measurements of a test subject including the grounding device of Examples 1-32, an electrodermal probe for contacting a second portion of the test subject, the electrodermal probe comprising a probe tip that contacts the second portion of the test subject.

Example 34 is a method of performing conductance testing using the bioelectric testing system of Example 33 for taking bioelectric measurements of a test subject including the grounding device of Examples 1-32 wherein the method comprises wetting a moistening media with a conductive fluid and placing the moistening media adjacent to the grounding plate and the apertures; placing the test subject's skin in contact with the grounding plate and the moistening media; and taking one or more bioelectric conductance measurements on one or more meridian points of the test subject by contacting the meridian point of the test subject with the electrodermal probe.

Example 35 is a method of performing conductance testing using the grounding device of Examples 1-32, wherein the method comprises placing test subject's skin in contact with a grounding plate (including moistening media) and two grounding segments, taking a first conductance reading between a first portion of the test subject's skin in contact with a first grounding segment and a second portion of test subject's skin in contact with the grounding plate, taking a second conductance reading between a first portion of the test subject's skin in contact with a first grounding segment, a second portion of test subject's skin in contact with the grounding plate, and a third portion of the test subjects hand in contact with the second grounding plate, comparing the first conductance reading with the second conductance reading to determine if the numbers are sufficiently similar or different from each other, remoistening the moistening media and repeating conductance testing if it is determined that the first measurement and the second measurement are different from each other, and performing conductance testing on meridian points of the test subject if it is determined that the first measurement and the second measurement are substantially similar to each other.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of pats so described and illustrated. The scope of the disclosure is to be defined by any claims appended hereto, any future claims submitted herein, and in different applications, and their equivalents.

What is claimed is:

1. A conductive device configured to contact skin of a test subject during bioelectric testing of the test subject, the conductive device comprising:
a first conductive portion that is electrically conductive;
one or more apertures formed through the first conductive portion; and
moistening media that applies a conductive fluid to the skin of the test subject through the one or more apertures.

2. The conductive device according to claim 1, wherein the conductive device is a grounding device for grounding the test subject.

3. The conductive device according to claim 1, further comprising:
a cover; and
a base;
wherein the cover is configured to alternate between an open position and a closed position relative to the base.

4. The conductive device according to claim 3, wherein the first conductive portion is disposed on the cover.

5. The conductive device according to claim 4, wherein the moistening media is disposed adjacent to the apertures in the first conductive portion of the cover and between the cover and the base.

6. The conductive device according to claim 5, wherein the cover comprises an openable and closeable compartment for holding the moistening media adjacent to the apertures of the first conductive portion.

7. The conductive device according to claim 3, wherein the base comprises one or more second conductive portions.

8. The conductive device according to claim 7, wherein the cover is in electrical communication with the base when the cover is in the closed position and the cover is electrically separated from the base when the cover is in the open position.

9. The conductive device according to claim 7, wherein the one or more second conductive portions are disposed on the base such that the one or more second conductive portions contact a palm of a hand of the test subject during the bioelectric testing; and
wherein the first conductive portion is disposed on the cover such that the first conductive portion contacts fingers and palm of the hand of the test subject during the bioelectric testing.

10. The conductive device according to claim 8, wherein the cover is in electrical communication with the base through a conductive contact on the base that contacts the first conductive portion of the cover.

11. The conductive device according to claim 10, wherein the conductive contact is spring-biased to press against the first conductive portion when the cover is in the closed position.

12. The conductive device according to claim 3, wherein the base comprises one or more of:
a battery to power the conductive device; and
an electrical connection to receive electricity from an outside power source to power the conductive device.

13. The conductive device according to claim 1, wherein the apertures are circular.

14. The conductive device according to claim 1, wherein the apertures are slits.

15. The conductive device according to claim 9, wherein the base further comprises a palm locating rib indicating where a test subject's palm may be placed on the conductive device.

16. The conductive device according to claim 1, wherein the first conductive portion is substantially plate-shaped.

17. The conductive device according to claim 1, wherein the first conductive portion is substantially bar-shaped.

18. The conductive device according to claim 3,
wherein the base comprises a pin;
wherein the cover comprises a knuckle that engages with the pin to form a hinge; and
wherein the cover rotates about the hinge to alternate between the open position and the closed position.

19. A bioelectric testing system for taking bioelectric measurements of a test subject, the bioelectric testing system comprising:
a grounding device for contacting a first portion of a test subject, the grounding device comprising:
a first conductive portion that is electrically conductive and contacts skin of the test subject;
one or more apertures formed through the first conductive portion; and
moistening media that applies a conductive fluid to the skin of the test subject through the one or more apertures; and
an electrodermal probe for contacting a second portion of the test subject, the electrodermal probe comprising:
a probe tip that contacts the second portion of the test subject.

20. A method for taking bioelectric measurements of a test subject with a bioelectric testing system comprising a grounding device comprising a first conductive portion that is electrically conductive and contacts skin of the test subject, one or more apertures formed through the first conductive portion, and moistening media that applies a conductive fluid to the skin of the test subject through the one or more apertures; and an electrodermal probe for contacting a second portion of the test subject, wherein the method comprises:
wetting a moistening media with a conductive fluid and placing the moistening media adjacent to the grounding plate and the apertures;
placing the test subject's skin in contact with the grounding plate and the moistening media; and
taking one or more bioelectric conductance measurements on one or more meridian points of the test subject by contacting the meridian point of the test subject with the electrodermal probe.
